(19) United States
(12) Patent Application Publication
(10) Pub. No.: US 2005/0009074 A1
(43) Pub. Date: Jan. 13, 2005

(54) Implantable monitor of vulnerable plaque and other disease states

(75) Inventor: David L. Thompson, Andover, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/884,249

(22) Filed: Jul. 1, 2004

Related U.S. Application Data
(60) Provisional application No. 60/485,153, filed on Jul. 7, 2003.

(51) Int. Cl. C12Q 1/68; G01N 33/53; A61K 49/00; A61B 5/00

(52) U.S. Cl. 435/6; 435/7.5; 600/300

(57) ABSTRACT

A method and implantable devices for monitoring a disease state in a patient. The method includes providing a monitor for C-reactive protein and implanting the monitor in the patient. At least one molecule binds to the C-reactive protein. A blood concentration of the C-reactive protein is determined based on the binding. A first implantable device includes a housing and a substrate including at least one molecule directed to the C-reactive protein. The first device further includes a detector adapted for measuring binding of the C-reactive protein to the molecule. A second implantable device includes implantable means for monitoring a C-reactive protein and means for binding the C-reactive protein. The second device further includes means for determining a blood concentration of the C-reactive protein based on the binding means.

### RELATED APPLIATIONS

This application claims the benefit of United States Provisional Patent Application 60/485,153 filed Jul. 7, 2003.

### TECHNICAL FIELD OF THE INVENTION

The present invention relates generally to C-reactive protein. More particularly, the invention relates to implantable monitoring strategies of vulnerable plaque associated with a blood vessel of a patient as well as other disease states.

### BACKGROUND OF THE INVENTION

Heart disease, specifically coronary artery disease, is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged changing the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap some of which are only two micrometers thick or less. The lipid pool is believed to be formed as a result of a pathological process involving low density lipoprotein (LDL), macrophages and the inflammatory process. The macrophages oxidize the LDL producing foam cells. The macrophages, foam cells, and associated endothelial cells release various substances, such as tumor necrosis factor, tissue factor and matrix proteinases, which result in generalized cell necrosis and apoptosis, pro-coagulation and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection (e.g., diagnosis and localization) and monitoring of vulnerable plaques. One strategy involves the measurement of temperature within a blood vessel. A localized increase in temperature is generally associated with the vulnerable plaque because of the tissue damage and inflammation. It has been observed that the inflamed necrotic core of the vulnerable plaque maintains a temperature of one or more degrees Celsius higher than that of the surrounding tissue. Measurement of these temperature differences within the blood vessel may provide means for detecting vulnerable plaque. Alternatively, numerous other physical properties, changes, factors, molecules, and the like specific to the vulnerable plaque may facilitate detection and monitoring.

Another strategy developed for the detection and monitoring of vulnerable plaque involves the use of radioactive tracers. An example of such a strategy is disclosed in U.S. Pat. No. 6,295,680 issued to Wahl et al. According to the Wahl Patent, an intravenous solution containing a radioactive tracer, which specifically accumulates in the vulnerable plaque, is administered to the patient. A miniaturized radiation detector is positioned within the patient's arterial lumen (e.g., endovascularly) for localized radioactivity imaging and detection. The radiation detector identifies and differentiates vulnerable plaque from inactive, stable plaque.

Other strategies for detecting and monitoring vulnerable plaque utilize imaging techniques, including endovascular and external approaches, utilizing any number of devices that can detect magnetic resonance, ultrasound, infra-red, near infra-red, fluorescence, visible light, radio waves, x-ray, etc. The endovascular approach may rely on an endoluminal device, such as a catheter, positioned adjacent the vulnerable plaque. The external approach may rely on a scanning device positioned outside the patient's body or inserted through an incision made in the patient. Energy pulses in the form of electromagnetic radiation or sound waves are directed toward the vulnerable plaque. Detectors are able to detect the reflected energy pulse from the vulnerable plaque, which is different from energy reflected from otherwise healthy vascular tissue. Such strategies may include the advantages of being non-invasive or at least minimally invasive and are capable of being performed in an outpatient setting.

Although the aforementioned strategies may provide effective vulnerable plaque detection, diagnosis, localization, and/or monitoring, they are relatively time consuming and typically cannot be performed outside of a clinical setting. It would be impractical to perform these procedures on a repeated basis so as to monitor a patient over a course of time. As such, it would be desirable to provide relatively easy and repeated monitoring of vulnerable plaque and/or other disease states over an extended time period.

C-reactive protein (CRP) is a plasma protein that is synthesized in the liver and consists of five identical, non-glycosylated polypeptide subunits that are noncovalently linked to form a disc-shaped pentamer with a molecular weight of about 125,000 Daltons. CRP is synthesized by hepatocytes in response to cytokines released into the liver by activated leukocytes. CRP is a prototypic acute phase protein that increases rapidly in concentration as a result of tissue injury, inflammation, or infection. The normal range of serum CRP is about 0.08-3 milligram (mg) per liter (l). However, CRP levels can increase between 100-1000-fold during an inflammatory response. Elevated serum levels of CRP are seen about 4-12 hours (h) after an inflammatory stimulus, and maximum levels are reached within 48-72 h. Generally, CRP levels will return to normal 5-10 days after remission of inflammation. Because the accumulation of CRP in serum closely parallels the course of inflammation and tissue injury, CRP has been used as a diagnostic tool to detect inflammation and to monitor the clinical course of various diseases. For example, CRP levels sometimes exceed 50 mg/l in rheumatoid arthritis, systemic lupus erythematosus (SLE), ulcerative colitis, Crohn's disease, acute pancreatitis, cardiac infarction, septicemia, bacterial infections including meningitis, some viral infections, pneumonia, tissue injury (e.g., burns, wounds, surgery, trauma, etc.), and other (inflammatory) conditions. Monitoring disease activity by measuring the serum concentration level of CRP has become a common practice in clinical chemistry.

CRP is also a risk indicator for coronary heart disease and vulnerable plaque, which involves the inflammatory response. Among the various prognostic markers of heart disease, such as serum amyloid A, soluble intercellular adhesion molecule type 1, interleukin-6, homocysteine, total cholesterol, LDL, apolipoprotein B-100, HDL, and ratio of total cholesterol to HDL, CRP is the strongest predictor of cardiovascular events. When apparently healthy adults are tested for CRP, the fourth quartile (upper 25%) of those tested have been shown to have over four times the risk of those in the first quartile (with a confidence level of 95%), a ratio significantly greater than those of each of the markers listed above.

Numerous strategies have been developed for the quantitative measurement of plasma CRP. One strategy involves the use of one or more monoclonal antibodies direct toward various CRP epitope(s). Examples of the monoclonal antibody strategy are disclosed in U.S. Pat. No. 5,358,852 to Wu, U.S. Pat. No. 5,5500,345 to Soe et al., and U.S. Pat. No. 6,406,862 issued to Krakauer. Other strategies for measuring plasma CRP utilize so-called, “high-sensitivity” CRP (hsCRP) detection methods. Automated analyzers on which tests for hsCRP can be performed are described in U.S. Pat. No. 6,548,646 to Ebrahim et al. and include the Dade Behring BN II Plasma Protein System (Dade Behring, Incorporated, Deerfield, Ill., USA), Abbott Laboratories IMx Immunoassay Analyzer (Abbott Laboratories, Abbott Park, Ill., USA), IMMULITE (Diagnostics Products Corporation, Los Angeles, Calif., USA), and IMMAGE (Beckman Coulter, Inc., Fullerton, Calif., USA). The Dade Behring BN II assay utilizes a monoclonal antibody on a polystyrene particle with fixed-time nephelometric measurements. The Abbott IMx assay is a two-site chemiluminescent enzyme immunometric assay with one monoclonal and one polyclonal anti-CRP antibody. The Beckman Coulter IMMAGE assay uses a polyclonal anti-CRP antibody on latex particles with rate nephelometric measurements. The detection limits for these assays range from 0.01 mg/L to 1.0 mg/L, and these instruments are calibrated for accuracy at CRP concentrations within these ranges, which are below those traditionally measured in clinical laboratories for less sensitive CRP assays.

The described strategies may allow for reliable measurement of serum CRP concentration and thus provide a predictive risk assessment for cardiovascular events and other disease states. However, the patient is generally required to provide a blood sample in a clinical setting followed by measurement of the CRP. As previously described, this may restrict the patient from obtaining repeated CRP measurements over a time course. As such, it would be desirable to provide an accurate measurement of blood CRP levels over a time course that would not restrict the patient to a clinical setting and/or drawing of repeated blood samples.

Accordingly, it would be desirable to provide implantable monitoring of vulnerable plaque and other disease states that would overcome the aforementioned and other disadvantages.

### SUMMARY OF THE INVENTION

A first aspect according to the invention provides a method of monitoring a disease state in a patient. The method includes providing a monitor for C-reactive protein and implanting the monitor in the patient. At least one molecule binds to the C-reactive protein. A blood concentration of the C-reactive protein is determined based on the binding.

A second aspect according to the invention provides an implantable device for monitoring a disease state in a patient. The implantable device includes a housing and a substrate including at least one molecule directed to the C-reactive protein. The device further includes a detector adapted for measuring binding of the C-reactive protein to the molecule.

A third aspect according to the invention provides an implantable device for monitoring a disease state in a patient. The implantable device includes implantable means for monitoring a C-reactive protein and means for binding the C-reactive protein. The device further includes means for determining a blood concentration of the C-reactive protein based on the binding means.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a flow chart of a method of monitoring a disease state in a patient, in accordance with the present invention;

FIG. 2 is a schematic view of an implantable device for monitoring a disease state in a patient, in accordance with one embodiment of the present invention;

FIG. 3 is a schematic view of a portion of an implantable device for monitoring a disease state in a patient, in accordance with another embodiment of the present invention;

FIGS. 4A and 4B are sequential schematic views of C-reactive protein (CRP) binding to a sensing component and subsequent detection of bound CRP, in accordance with the present invention; and

FIG. 5 is a schematic view of an implantable device for monitoring a disease state implanted within a patient, in accordance with the present invention.

### DESCRIPTION OF THE PRESENTLY PREFERRED EMBODIMENTS

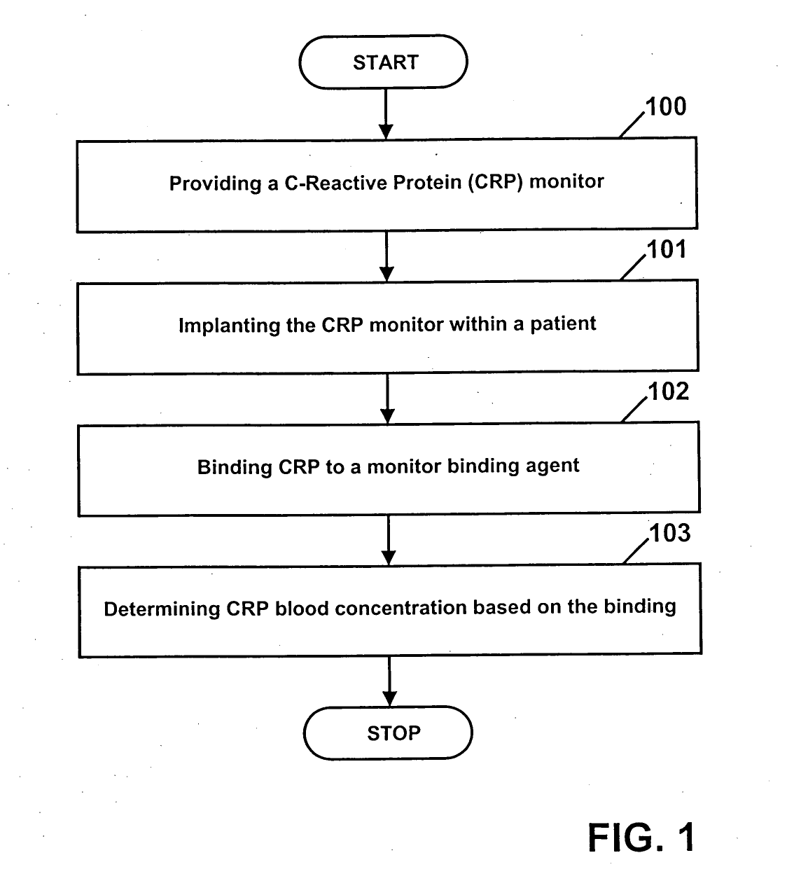

Referring to the drawings, wherein like reference numerals refer to like elements, FIG. 1 is a flow chart of a method of monitoring a disease state in a patient, in accordance with the present invention. In the following description, the term “monitoring” and its derivates refer to a process of detecting and/or observing a disease state. In one embodiment of the present invention, the disease state may be a condition such as vulnerable plaque, rheumatoid arthritis, systemic lupus erythematosus, ulcerative colitis, Crohn's disease, acute pancreatitis, cardiac infarction, septicemia, infection, meningitis, pneumonia, tissue injury, burn, wound, trauma, and an inflammatory condition. In another embodiment, the disease state may be a variety of conditions or states that correlate with altered (e.g., elevated or decreased) C-reactive protein (CRP) levels in the blood. As such, the determination of CRP concentration may be utilized to detect and/or monitor the disease state.

The following description relates primarily to the detection and monitoring of vulnerable plaque. A vulnerable plaque is distinguishable from other types of plaque, including hard plaques, by the presence of a fibrous cap. The vulnerable plaque fibrous cap retains a pool of lipids and other contents, which may be released into the blood vessel upon rupture. The released contents may form emboli that can lodge in a blood vessel thereby posing a risk to the patient. Vulnerable plaques, unlike hard plaques, are generally non-occlusive and as such, may not produce angina.

Those skilled in the art will recognize that although the present invention is described primarily in the context of detecting vulnerable plaque while using specific implantable devices, the inventor contemplates alternative devices and methods of application and the monitoring of numerous other disease states. Any number of devices capable of performing the prescribed function(s) may be adapted for use with the present invention. Furthermore, the detection and monitoring strategies are not limited to the described methodologies and disease states. Numerous modifications, substitutions, and variations may be made to the devices and methods while providing effective disease state monitoring consistent with the present invention.

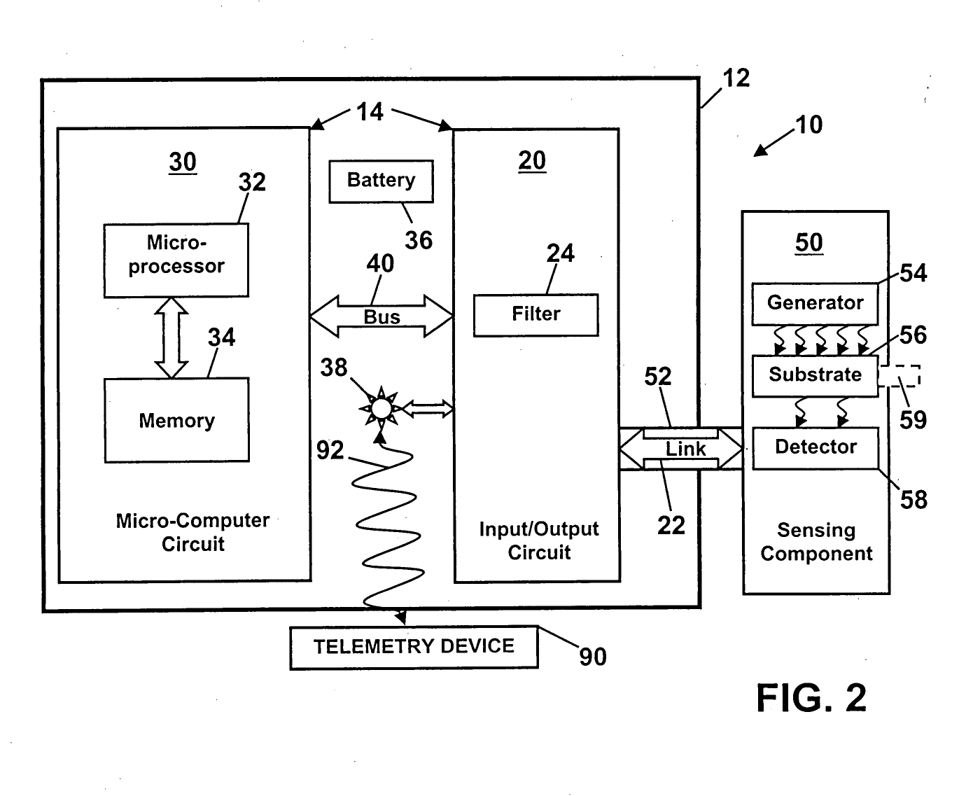

As shown in FIG. 1, monitoring of vulnerable plaque may begin by providing an implantable CRP monitor (step 100). In one embodiment, as shown in FIG. 2, monitor 10 may include a housing 12 manufactured from a biocompatible material, such as stainless steel, titanium, tantalum, ceramic, a composite, a polymer, and the like. Preferably, the housing 12 provides a hermetically sealed enclosure from external fluids thereby protecting all or part of monitor circuitry 14. Circuitry 14 may comprise electronically coupled input/output circuit 20, micro-computer circuit 30, which may include one or more digital microprocessors programmed to process a plurality of input signals in a stored algorithm and generate output signals, and sensing component 50. The methods, algorithms, and determinations (e.g., calculations and estimations) of the present invention, including those based on equations or value tables, may be performed by a device such as the micro-computer circuit 30.

Input/output circuit 20 may communicate with the sensing component 50 via an electronic and/or optical link 22 and provide output indicative of vulnerable plaque status. Micro-computer circuit 30, specifically, a micro-processor 32 may perform determinations based on the received input from the input/output circuit 20. Input/output circuit 20 and micro-computer circuit 30 may be electronically coupled via a data communications bus 40 and correspond to the input/output circuit and micro-computer circuit disclosed in U.S. Pat. No. 6,514,195 issued to Ferek-Petric or the microcomputer circuit disclosed in U.S. Pat. No. 5,312,453 issued to Shelton et al. Computer usable medium, value tables, and other data associated with the present invention may be programmed, read, and stored into/from a microprocessor memory portion 34 (e.g., ROM, RAM, and the like). Such information may be accessible to executing algorithms (e.g., programs) associated with the present invention.

Monitor 10 may be powered by an appropriate implantable battery power source 36 in accordance with common practice in the art. For the sake of clarity, the coupling of battery power to the various components of the monitor 10 is not shown in the Figures. Monitor 10 may be programmed by means of an external programming unit, such as a telemetry device 90. One such programmer is the commercially available Medtronic Model 9790 programmer, which is microprocessor-based and provides a series of encoded signals to the Input/output circuit 20, typically through a programming head including an antenna 38 that transmits or telemeters radio-frequency (RF) encoded signals 92. Such a telemetry system is described in U.S. Pat. No. 5,312,453 issued to Wyborny et al. The programming strategy disclosed in the Wyborny '453 patent is identified herein for illustrative purposes only. Furthermore, the antenna 38 signal may be coupled to the Internet directly or through the telemetry device 90 thereby providing remote communication means with the monitor 10. Any of a number of suitable programming and telemetry methodologies known in the art may be employed so long as the desired information is transmitted to and from the monitor 10. Telemetry device 90 may be a patient alarm that, as described below, may communicate the disease state to the patient. The specific embodiments of the input/output circuit 20, programming head including antenna 38 presented herein are shown for illustrative purposes only, and are not intended to limit the scope of the present invention.

Analog signal processing may be provided by a filter 24 for some of the input signals received by input/output circuit 20. For example, signals received by the input/output circuit 20 from the sensing component 50 may be filtered to eliminate signal “noise” that may serve to interfere with appropriate determination of vulnerable plaque status. Those skilled in the art will recognize that the input/output circuit 20, micro-computer circuit 30, and sensing component 50 described herein may vary and that numerous such devices may be adapted for use with the present invention.

Sensing component 50 may include one or more leads 52 extending there-from allowing sampling of patient blood distally from the monitor 10. As such, the monitor 10 may be implanted within a patient at a suitable locus remote from the blood vessel(s) where monitoring occurs. In one embodiment, sensing component 50 may include a generator 54 for emitting electromagnetic energy on a portion of a substrate 56. The electromagnetic energy that interacts with the substrate 56 portion may be detected by a detector 58. As described below, the type and/or degree of interaction of the electromagnetic radiation with the substrate 56 portion are based on blood concentration of CRP. As such, this property may be exploited to monitor vulnerable plaque (or other disease states). Patient blood may be sampled via a port 59 allowing flow to and from the substrate 56 portion.

In one embodiment, the generator 54 may produce (ultra)sonic energy or any sub-spectrum of electromagnetic energy including, but not limited to, radio wave radiation, microwave radiation, x-ray radiation, beta radiation, and the like for emission on the substrate 56 and subsequent detection by an appropriate detector 58. Generator 54 and detector 58 may be positioned adjacent the substrate 56 and distally coupled to the monitor 10 via the link 52 comprising one or more wires, fiber optic members, and the like.

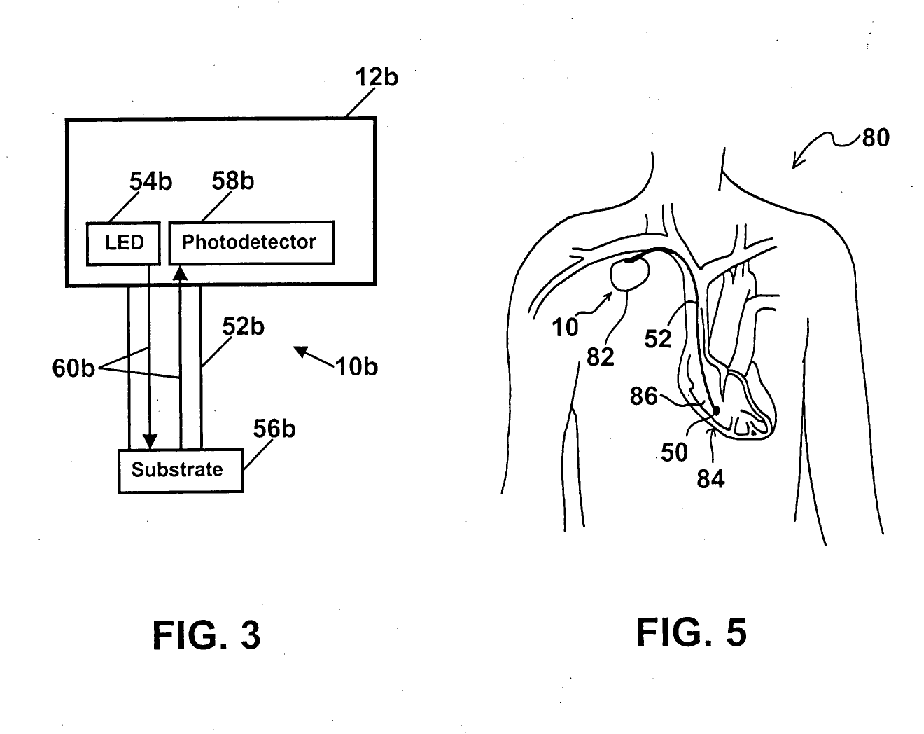

In another embodiment shown in FIG. 3, the generator 54b may be a light emitting diode (LED) capable of producing light energy (e.g., infra-red radiation, near infra-red radiation, visible light radiation, ultraviolet radiation, etc.) whereby the resulting light energy (including fluorescence radiation) may be detected by a photodetector 58b. Specifically, the generator 54b may emit infra-red or near-infra-red radiation at a wavelength of about 700 to 3,000 nanometers, such as that produced by an optical coherence tomography (OCT) device. Generator 54b, substrate 56b, and detector 58b may be positioned at numerous locations relative to the monitor 10b. For example, as shown, the LED generator 54b and photodetector 58b may be positioned within monitor housing 12b thereby minimizing the size of lead 52b. Substrate 56b may be distally positioned on the lead 52b and coupled to the LED generator 54b and photodetector 58b via one or more fiber optic members 60b. As another example (not shown), the generator, substrate, and detector may each be positioned within the monitor housing wherein blood may be circulated between the monitor and blood sampling site via a plurality of tubes. Those skilled in the art will recognize that the configuration, arrangement, geometry, positioning, coupling, and operation of the sensing component may be varied while still providing effective disease state monitoring.

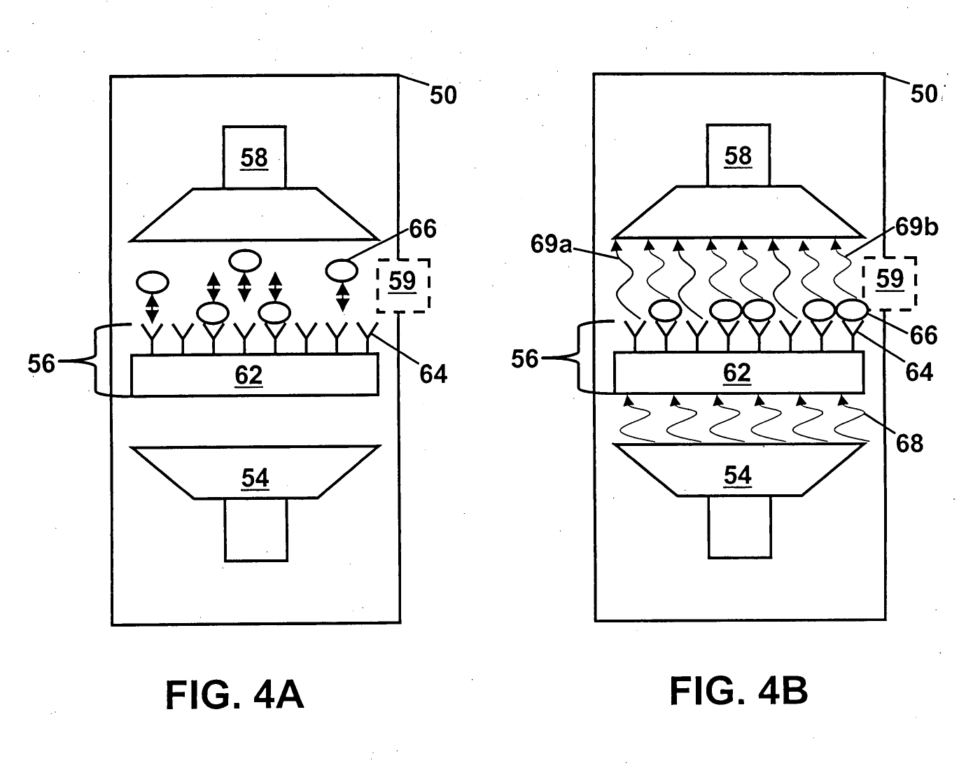

Referring to FIGS. 4A and 4B, substrate 56 may include an underlying surface 62 and one or more, in this case one, type of binding agent 64 disposed thereon. Underlying surface 62 may be manufactured from any number of biocompatible materials capable of supporting and retaining the binding agent 64. Exemplary underlying surface 62 materials include, but are not limited to, polypropylene, polyethylene, polyvinyl chloride, plastic, and the like. Underlying surface 62 may include a variety of geometries, sizes, and surfaces. Furthermore, the underlying surface 62 may be treated with one or more reagents for retaining and/or optimizing function of the binding agent 64. The use of substrates including an underlying surface, one or more binding agents, reagents, and/or other components are known in the art and are commercially available as part of some enzyme linked immunosorbent assay (ELISA) kits.

In one embodiment, the binding agent 64 may be an antibody specific for CRP 66. The NycoCard® kit by Axis Shield includes a CRP 66 antibody that may be adapted for use as the binding agent 64. Underlying surface 62 may be treated with a reagent of coating stabilizer and blocking buffer formulated to improve the stability and function of solid phase proteins (antigens, antibodies, etc.) thereby enhancing binding. The enhanced binding is a consequence of the reagent's stabilizing effect on the tertiary structure of proteins on the solid phase. Improved maintenance of tertiary structure results in optimal antigenic function because there is less denatured folding of the protein to mask antigenic regions.

In another embodiment, the binding agent may be a C-reactive protein binding protein (CRPBP). In yet another embodiment, the binding agent may include a micro- or nano-particle. The micro- and nano-particles may be a sphere or other geometry about 0.5 to 10.0 micrometers and 10 to 200 nanometers in diameter, respectively, and comprised of a protein shell filled with air/gas. Such particles are known in the art and may be a polymer composite, a powder, and/or a tube of the fullerene family of carbon molecules as known in the art. Such detection agents may be manufactured from metals, alloys, polymers, or organic materials and may include a surface coating with affinity for CRP. Detection of the particles bound to CRP may be achieved when the particle absorbs one wavelength of light and emits light radiation (e.g., fluorescence) a different wavelength (i.e., upon CRP binding or release). The particle may be differentially sized to provide a unique light fluorescence wavelength.

In yet another embodiment, the binding agent may be one or more molecules such as another immunoglobulin (e.g., monoclonal and/or polyclonal), an Fc class receptor, a major histocompatability complex (MHC) molecule (e.g., class I and class II), a phosphocoline, a CD molecule (e.g., CD3, CD4, CD8, CD19, CD16 and CD56), a polysaccharide, a polycation, a binding molecule, a binding protein, a membrane protein, a polynucleotide (e.g., DNA, RNA, single-stranded, double-stranded, triple-stranded), an antisense polynucleotide, a modified polynucleotide, a biotinylated molecule, and the like for binding to C-reactive protein present within the blood stream.

Monitor is implanted within a patient (step 101). In one embodiment shown in FIG. 5, the monitor 10 may be implanted within a patient 80 at a predetermined implant site 82. Patient implant site 82, which in this case is adjacent the heart 84, may allow sampling of blood for determining a blood concentration of CRP. Specifically, the monitor 10 may be implanted within the patient 80 chest cavity in a manner similar to, for example, cardiac pacemakers wherein the procedure is performed under surgical conditions by a physician. Depending on the configuration of the monitor 10, the lead 52 may extend from the monitor 10 wherein the sensing component 50 is positioned within a chamber 86 of the heart 84 providing CRP level measurement therein. In another embodiment, the patient 80 implant and blood sampling sites may be any site that may provide for effective operation of the monitor 10 and benefit to the patient 80. For example, the lead may be positioned in the ostium of the coronary sinus, the great vein or distal coronary veins to monitor the venous outflow of the complete heart muscle. It is important to note that numerous implant and blood sampling sites (e.g., blood vessels) other than the ones illustrated and described may be used with the present invention.

During operation of the monitor, the binding agent may bind CRP (step 102). In one embodiment shown in FIG. 4A, the sensing component 50 binding agent 64 may reversibly bind CRP 66, as shown by double arrows, in proportion to its blood concentration, which may be sampled via the port 59. Relative increases or decreases in CRP 66 concentration may result in commensurate changes in binding level. The half-life of CRP 66 is relatively short at about 20 hours and is not influenced by age, liver or kidney function, or pharmacotherapy. As such, CRP 66 bound to the binding agent 64 may quickly degrade/disassociate thereby allowing for rapid and repeated determinations of blood CRP 66 concentration.

The blood concentration of CRP is then determined based on the binding level (step 103). The blood concentration of CRP may be determined continuously or, alternatively, intermittently at discrete intervals (i.e., to conserve battery power), such as 10 samples per second (sps), 1 sps, 10 samples per minute (spm), 1 spm, 1/10 spm, 1/60 spm, etc. In one embodiment shown in FIG. 4B, the generator 54 may emit light energy 68 on the substrate 56, specifically, the binding agent 64. The light energy interacts with the binding agent 64 differently depending on its CRP 66 binding state and the resulting light energy 69a, 69b may be detected by the photodetector 58. For example, light energy emitted at one wavelength 68 may shift to a second wavelength 69a after refraction from an unbound binding agent or shift to a third wavelength 69b after refraction from a bound binding agent-CRP 66 complex. As such, the relative intensity of the second wavelength 69a to the third wavelength 69b may be proportional to blood CRP 66 concentration.

As another example, light energy emitted on the binding agent may fluoresce from bound binding agent-CRP complexes thereby providing another strategy for determining blood CRP concentration. Likewise, sonic energy emitted on the binding agent from a sonic generator may resonate differently depending on the binding state (i.e., different resonant frequencies); other forms of electromagnetic radiation (e.g., radio wave radiation, microwave radiation, infra-red radiation, near infra-red radiation, visible light radiation, ultraviolet radiation, x-ray radiation, beta radiation, etc.) may interact differently depending on the binding agent 64 binding state.

Detector 58 may then detect the resulting light energy thereby providing means for estimating the level of CRP 66 binding. The monitor micro-computer circuit may receive information from the detector 58 via the input/output circuit and determine CRP 66 concentration through the use of equations, value tables, and the like. For example, a running average may be calculated and subsequent measured values may be compared to the average to monitor sudden increases (e.g., such as a 100-1000-fold increase during the 4-12 hour onset of an inflammatory stimulus). In one embodiment, these determinations may be communicated to a clinician via the telemetry feature. The telemetry feature may be coupled to an external indicator box/transponder 90 for direct reading and/or communication through the Internet to a monitoring facility or other means thereby allowing remote monitoring of a given disease state.

In another or the same embodiment, the patient may monitor the external indicator 90 and may be additionally provided with an automatic patient alarm as an indication of a rapid increase in CRP levels, which may provide visual or audio indications of the disease state. In some instances, the patient may be alerted once the CRP level reached a predetermined threshold level. These levels may be preset or tailored/programmed to a patient specific need. Depending upon the levels detected, patient instructions may include directions to call the patient's clinician and/or presenting themselves at a hospital emergency room.

While the embodiments of the invention disclosed herein are presently considered to be preferred, various changes and modifications may be made without departing from the spirit and scope of the invention. The devices and methods of the present invention are not limited to any particular design, configuration, implantation, or sequence. Specifically, disease state monitoring and the devices for achieving the same may vary without limiting the utility of the invention. For example, numerous implantable monitors may achieve monitoring of disease states. Furthermore, the specific diseases that correlate with blood CRP concentration and can therefore be monitored according to the present invention may vary.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A method of monitoring a disease state in a patient, the method comprising:
providing a monitor for C-reactive protein;
implanting the monitor in the patient;
binding at least one molecule to the C-reactive protein; and
determining a blood concentration of the C-reactive protein based on the binding.

2. The method of claim 1 wherein the disease state comprises at least one condition selected from a group consisting of vulnerable plaque, rheumatoid arthritis, systemic lupus erythematosus, ulcerative colitis, Crohn's disease, acute pancreatitis, cardiac infarction, septicemia, infection, meningitis, pneumonia, tissue injury, burn, wound, trauma, and an inflammatory condition.

3. The method of claim 1 wherein the at least one molecule comprises at least one molecule selected from a group consisting of a C-reactive binding protein, an immunoglobulin, an Fc class receptor, a major histocompatability complex molecule, a phosphocoline, a CD, a polysaccharide, a polycation, a binding molecule, a binding protein, a membrane protein, a polynucleotide, an antisense polynucleotide, a modified polynucleotide, and a biotinylated molecule.

4. The method of claim 1 wherein the binding of the at least one molecule to the C-reactive protein comprises a reversible binding.

5. The method of claim 1 wherein determining the blood concentration of the C-reactive protein comprises one or more determinations selected from a group consisting of repeated determinations, continuous determinations, intermittent determinations, and running average determinations.

6. The method of claim 1 wherein determining the blood concentration of the C-reactive protein comprises detecting electromagnetic radiation or sonic energy.

7. The method of claim 6 wherein the electromagnetic radiation is selected from a group consisting of radio wave radiation, microwave radiation, infra-red radiation, near infra-red radiation, visible light radiation, ultraviolet radiation, x-ray radiation, beta radiation, and fluorescence radiation.

8. The method of claim 1 further comprising applying electromagnetic radiation or sonic energy directed at the at least one molecule.

9. The method of claim 8 wherein the electromagnetic radiation is selected from a group consisting of radio wave radiation, microwave radiation, infra-red radiation, near infra-red radiation, visible light radiation, ultraviolet radiation, x-ray radiation, beta radiation, and fluorescence radiation.

10. The method of claim 1 further comprising communicating the determined blood concentration of the C-reactive protein through the Internet.

11. The method of claim 1 further comprising communicating the determined blood concentration of the C-reactive protein to the patient.

12. An implantable device for monitoring a disease state in a patient, the device comprising:
a housing;
a substrate including at least one molecule directed to the C-reactive protein; and
a detector adapted for measuring binding of the C-reactive protein to the molecule.

13. The device of claim 12 wherein the disease state comprises at least one condition selected from a group consisting of vulnerable plaque, rheumatoid arthritis, systemic lupus erythematosus, ulcerative colitis, Crohn's disease, acute pancreatitis, cardiac infarction, septicemia, infection, meningitis, pneumonia, tissue injury, burn, wound, trauma, and an inflammatory condition.

14. The device of claim 12 wherein the substrate comprises an underlying surface including the at least one molecule disposed thereon.

15. The device of claim 12 wherein the at least one molecule comprises a particle.

16. The device of claim 15 wherein the particle comprises a micro-particle of about 0.5 to 10.0 micrometers in diameter.

17. The device of claim 15 wherein the particle comprises a nano-particle of about 10 to 200 nanometers in diameter.

18. The device of claim 12 wherein the molecule comprises at least one molecule selected from a group consisting of a C-reactive binding protein, an immunoglobulin, an Fc class receptor, a major histocompatability complex molecule, a phosphocoline, a CD, a polysaccharide, a polycation, a binding molecule, a binding protein, a membrane protein, a polynucleotide, an antisense polynucleotide, a modified polynucleotide, and a biotinylated molecule.

19. The device of claim 12 wherein the detector measures the binding of the C-reactive protein to the at least one molecule continuously or intermittently.

20. The device of claim 12 wherein the detector detects electromagnetic radiation or sonic energy.

21. The device of claim 20 wherein the electromagnetic radiation is selected from a group consisting of radio wave radiation, microwave radiation, infra-red radiation, near infra-red radiation, visible light radiation, ultraviolet radiation, x-ray radiation, beta radiation, and fluorescence radiation.

22. The device of claim 12 further comprising a generator operably coupled to the detector for applying electromagnetic radiation or sonic energy directed to the at least one molecule.

23. The device of claim 22 wherein the electromagnetic radiation is selected from a group consisting of radio wave radiation, microwave radiation, infra-red radiation, near infra-red radiation, visible light radiation, ultraviolet radiation, x-ray radiation, beta radiation, and fluorescence radiation.

24. The device of claim 12 wherein the monitor is operably coupled to the Internet.

25. The device of claim 12 further comprising at least one lead extending from the body and adapted for positioning at a blood sampling site.

27. The device of claim 12 further comprising:
an input/output circuit operably coupled to the detector; and
a micro-computer circuit operably coupled to the input/output circuit for determining a blood concentration of the C-reactive protein based on the measured binding of the C-reactive protein to the at least one molecule.

28. The device of claim 27 wherein the micro-computer circuit determines a running average of the C-reactive protein blood concentration.

29. The device of claim 12 further comprising a patient alarm operably coupled to the monitor for indicating the monitored disease state.

30. The device of claim 26 wherein the patient alarm comprises at least one predetermined threshold level.

31. An implantable device for monitoring a disease state in a patient, the device comprising:
implantable means for monitoring a C-reactive protein;
means for binding the C-reactive protein; and
means for determining a blood concentration of the C-reactive protein based on the binding means.

32. The device of claim 31 further comprising means for applying electromagnetic radiation or sonic energy directed at the molecule.

33. The device of claim 31 further comprising means for communicating the determined blood concentration of the C-reactive protein external to the patient.
